(19) United States
(12) Plant Patent Application Publication
(10) Pub. No.: US 2009/0235417 P1
(43) Pub. Date: Sep. 17, 2009

(54) Oncidium heaven scent 'sweet baby'

(50) ;

(76) Inventor: James William McCully, Hilo, HI (US)

Correspondence Address: JAMES WILLIAM MCCULLY, 2146 KAUMANA DRIVE, HILO, HI, 96720, US

(21) Appl. No.: 12/009,396

(22) Filed: Jan. 18, 2008

(51) Int. Cl. A01H 5/00

(52) U.S. Cl. PLT/311

(57) ABSTRACT

A new variety of orchid plant of the Oncidiinae Intergenerics, Oncidium, named Oncidium Heaven Scent ‘Sweet Baby’, distinguished particularly by a compact size, multiple spikes, sweet fragrance, many burgundy/pink flowers nicely shaped and arranged pleasingly on a panicle that grows quickly to sexual maturity.

### CROSS-REFERENCE TO RELATED APPLICATIONS

Not Applicable

### STATEMENT REGARDING FEDERALLY SPONSORED RESEARCH OR DEVELOPMENT

Not Applicable

### LATIN NAME OF THE GENUS AND GREX OF THE PLANT CLAIMED

The Latin name of the genus and grex of the plant claimed is Oncidium Heaven Scent.

### VARIETY DENOMINATION

The present invention comprises a new and distinct cultivar of Oncidium orchid, and hereinafter referred to by the cultivar name Oncidium Heaven Scent ‘Sweet Baby’.

### BACKGROUND OF THE INVENTION

Oncidium comprises a genus of approximately 400 species from the tropical and subtropical Americas. Oncidium orchids are primarily epiphytic or lithophytic with a minor portion being terrestrial. All species exhibit sympodial growth and vary greatly in other morphology and size.

Oncidium breeding is typically done from sexual methods. Asexual propagation of Oncidiinae is often done in aseptic tissue culture from apical or axillary shoots.

The new cultivar was discovered by the Inventor within the progeny of a cross made by the Inventor on Sep. 3, 2001. The Inventor selected Oncidium Heaven Scent ‘Sweet Baby’ as a single plant from a population of over 50 plants grown by James McCully Orchid Culture Inc. dba Mauna Kea Orchids in Hilo, Hi., a company owned by the Inventor. Oncidium Heaven Scent ‘Sweet Baby’ was submitted by the Inventor to a commercial tissue culture laboratory, Bangkok Flower Centre, Bangkok, Thailand on Jun. 7, 2005, for propagation through aseptic tissue culture technique. A quantity was produced for evaluation and has demonstrated that the unique combination of characteristics as herein disclosed for the new cultivar are firmly fixed and are retained through successive generations of asexual reproduction.

Other seedlings from the same cross that was used to produce Oncidium Heaven Scent ‘Sweet Baby’ have been commercially available from others. The characteristics of Oncidium Heaven Scent ‘Sweet Baby’ are clearly distinguishable from the characteristics of those other plants. Those other plants were sold by James McCully Orchid Culture without specific epithet to individuals and other nurseries. To the Inventor's knowledge, the only other named clone of the cross is Oncidium Heaven Scent ‘Sweet Pea’ and is owned by the Inventor. Plants of the grex Oncidium Heaven Scent are sold as plants of the grex without specific epithet.

### BRIEF SUMMARY OF THE INVENTION

The following traits have been repeatedly observed and are determined to be basic characteristics of the new cultivar which in combination distinguish this Oncidium orchid as a new and distinct cultivar:

Flowers of the new cultivar are burgundy in color on the sepals and petals. The labellum is dark pink with a burgundy isthmus fading to a lighter pink color with irregular white spotting around the bottom edge. The callus of the lip swells into small tubercles, 1 to 2 mm long sometimes with white tips. The column is burgundy on the upper surface and white on the low surface, measuring 0.6 cm long by 0.6 cm wide. The column wings are narrowly lamellate. The anther cap is white with irregular burgundy spots overlaying the tip. The natural horizontal spread is 3.7 centimeters (cm); natural vertical spread is 4.4 cm; dorsal sepal width 0.7 cm; dorsal sepal length 1.7 cm; petal width 0.7 cm; petal length 1.7 cm; lateral sepal width 0.6 cm; lateral sepal length 2.3 cm; labellum width 2.0 cm; labellum length 2.3 cm. There are around 25-35 flowers on each spike on the first bloom depending on the plant's environment.

Inflorescence is a basal panicle, approximately 45 to 72 cm tall with 5 to 9 alternate lateral branching. The branching of the panicle starts at leaf level and the branches vary in length, shortening towards the top. The size of the inflorescence is appropriate to the overall size of the plant and typical pot size for this cultivar.

There are generally four to five leaves on the mature psuedobulb, two or three from the apex and two from the base of the psuedobulb. The apical leaves measure 25 to 43 cm and are 3.5 to 4 cm wide. The leaves from the base of the pseudobulb measure up to 30 cm and up to 4 cm wide. Axillary leaves, or bracts, are sometimes present below the leaves at the base of the psuedobulb. When present, there is usually one on either side of the pseudobulb and measure 7 to 13 cm long. The shape of the leaf is lanceolate acuminate and semi-conduplicate at the base. The texture and substance is smooth, waxy and thin. The plants of Oncidium Heaven Scent ‘Sweet Baby’ grow to maturity and flower in approximately 18 months.

Plants of the new cultivar have not been observed under all possible environmental conditions. The phenotype may vary significantly with variations in the environment such as temperature, light intensity, and day length, without however any change in genotype.

Plants of the new cultivar differ primarily from the plants of the parent cultivars in flower color, fragrance, panicle size, rate of growth and speed to maturation. Perhaps the closest commercial comparison to the new cultivar can be made to seedling-derived Oncidium orchids which are genetically heterogeneous, and typically lack uniformity in growth, vigor, plant habit, and flower quality. Since this reference point has inconsistent characteristics, a direct comparison for Oncidium Heaven Scent ‘Sweet Baby’ is not available. The new cultivar is a single genotype asexually propagated via tissue culture; thus, its combined horticultural characteristics listed above are uniform and predictable.

### BRIEF DESCRIPTION OF THE DRAWINGS

The drawings show the colors of the new variety as nearly true as possible with an illustration of this type. In the drawings:

FIG. 1 is a view of the entire plant to reveal the growth habit and general characteristics with its flowers, which is typical of this new variety.

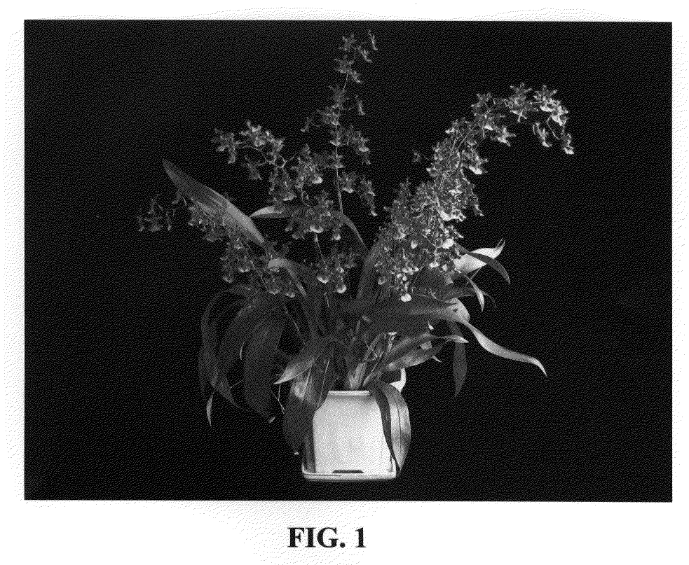

FIG. 2 is a close up view of the flower to show its shape.

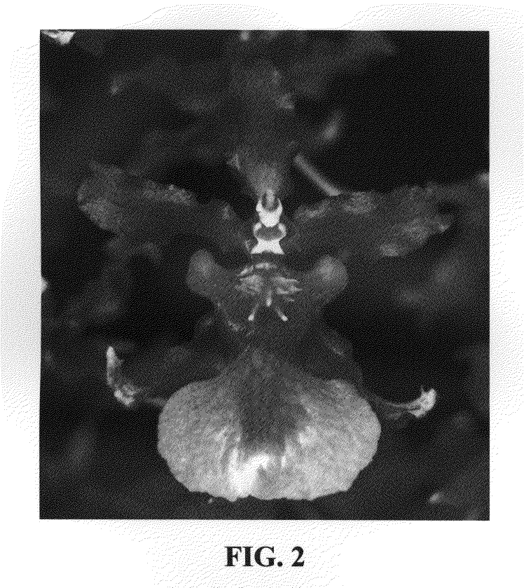

### DETAILED BOTANICAL DESCRIPTION

All color references are measured against the Pantone® Color System. Colors and numerical measurements are approximate as plant growth and development depends on environmental conditions and cultural practices such as light level, temperature, water quality, fertilization formula and rate, among others, without, however, any variance in genotype.

Plants used for this description are 1 to 2 years in vivo and grown in 3.3 inch, square black plastic pots, grown in a poly-carbonate covered greenhouse in Hilo, Hi., where day temperatures range from 72 to 88 degrees and night temperatures range from 56 to 72 degrees Fahrenheit. Light levels are between 1500 to 2500 foot candles. This information reflects the annual variations for the area.
Botanical classification: Oncidium Heaven Scent ‘Sweet Baby’
Parentage: Seedling selected from the cross of the following:
   Seed parent.—Oncidium Ruffles.
   Pollen parent.—Oncidium Sharry Baby.
Propagation:
   Type.—Asexual propagation by aseptic tissue culture through axillary shoot initiation.
   Time to initiate and elongate roots in-vivo.—about 45 days.
   Time to produce fully rooted young plant.—about 180 days.
Root description: Velamen covered roots, approximately 1.5 mm in diameter, white in color with yellow to green growing tip. The exact shades of white and yellow/green vary greatly with minimal changes in environmental conditions.
Plant description: Under appropriate growing conditions, plants of the new cultivar attain a mature size of about 43 cm in height (top of leaf plane) and about 4 cm in width.
Foliage description:
   Arrangement.—Sympodial growth habit.
   Quantity of leaves per growth.—Each sympodial growth has shown four to five leaves, two to three on the apex of the pseudobulb and two from the axis at the base of the pseudobulb.
   Leaf length.—The larger apical leaves measure up to 43 cm in length. The axillary leaves at the base of the psuedobulb may be as short as to appear to be a bract and as long as 13 cm.
   Leaf width.—The width of the largest leaf measures up to 4 cm.
   Leaf shape.—The shape is lanceolate acuminate, texture and substance is smooth, waxy and thin.
   Pseudobulb shape.—The pseudobulb shape is a laterally compressed ovoid.
   Pseudobulb length.—The pseudobulb is approximately 7 to 12 cm long.
   Psuedobulb width.—The pseudobulb is approximately 3.5 to 6.5 cm wide and 1.5 to 2.0 cm thick.
   Foliage color.—Under the described growing conditions, the foliage color on both sides of the leaves is similar to Pantone® 371UP (green) of the Pantone® Color System. There is no vein color distinction. The pseudobulb can vary from Pantone® 371UP (green) to Pantone® 377UP (green).
Flower/inflorescence description:
   Description.—Panicle, approximately 45 cm on a first bloom plant and has 5 to 9 alternate side branches. The branches start at leaf level and become shorter near the panicle apex. The size of the inflorescence is appropriate to the overall size of the plant and typical pot size for this cultivar.
   Dimensions.—Flower dimensions are: natural horizontal spread is 3.7 cm; natural vertical spread is 4.4 cm; dorsal sepal width is 0.7 cm; sepal length is 1.7 cm; petal width is 0.7 cm; petal length is 1.7 cm; lateral sepal width is 0.6 cm; lateral sepal length is 2.3 cm; labellum width is 2.0 cm; labellum length is 2.3 cm.
   Coloration.—The dominant flower color appears on the sepals and petals and is Pantone® 7428C (Burgundy). The lip is Pantone® 7433PC (Dark pink), with the isthmus of the lip having a darker patch of burgundy, Pantone® 7435PC. Towards the bottom of the lip, the color fades into a lighter pink, Pantone® 7431PC, sometimes with irregular white spotting (Pantone® 7527C). The column is burgundy, Pantone® 7428C, on the upper surface and the underside of the column is white (Pantone® 7527C). The anther cap is white, Pantone® 7527C, with irregular burgundy (Pantone® 7428C) markings on the tip. The pollinia are completely concealed by the anther cap, and if it is removed the pollinia may be extracted. The pollinia are yellowish orange, Pantone® 130C, but can range in shades of yellow as the plant ages.
Quantity of flowers and time to flower:
   Flower longevity.—The flowers can last up to 7 weeks on the plant with good environmental conditions. A first bloom plant will produce 25 to 35 flowers depending on environmental conditions.
   Natural flowering season.—The plant has exhibited free flowering although the dominant flowering season is fall/winter.
Fragrance: A strong, sweet fragrance has been detected by the Inventor.
Reproductive organs: The stamens, style and stigmas are fused into a single short structure called the column, possessing one terminal anther with pollen grains united to form pollinia, which are covered by an anther cap. The stigma is located under the column behind the pollinia. The ovary is inferior, with three carpels being present.
   Column.—The column is erect with narrowly lamellate wings on either side of the stigma, 6 millimeters (mm) wide by 6 mm long.
   Pollinia.—Two oval masses of pollen are present, about 1 mm in diameter.
   Stigma.—The stigma is concave, round, with a high gloss, and sticky.
   Ovary.—The ovary is about 2.5 cm long by 1.5 mm in diameter.
   Seed.—Seed production has not been observed.
Disease resistance: Resistance or susceptibility to known pathogens of Oncidium has not been observed on plants grown under commercial production conditions.
General observations: Plants of Oncidium Heaven Scent ‘Sweet Baby’ produce a pleasing arrangement of many burgundy/pink flowers on a panicle that is desirable in size for the size of the plant. The flowers are heavily scented, long lasting and nicely shaped. The plant normally produces multiple spikes and grows quickly to sexual maturity.

What is claimed is:

1. A new and distinct variety of orchid plant named Oncidium Heaven Scent ‘Sweet Baby’, substantially as illustrated and described herein.
